(19) United States
(12) Patent Application Publication
(10) Pub. No.: US 2008/0208481 A1
(43) Pub. Date: Aug. 28, 2008

(54) AUTOMATIC ANALYZER

(76) Inventors: Toshiro NAKANO, Mito (JP); Hitoshi Otake, Hitachinaka (JP)

Correspondence Address: Mattingly Stanger and Malur PC, Suite 370, 1800 Diagonal Rd, Alexandria, VA, 22314, US

(21) Appl. No.: 12/037,614

(22) Filed: Feb. 26, 2008

(30) Foreign Application Priority Data
Feb. 28, 2007 (JP) 2007-048353

(51) Int. Cl. G01N 33/48; G01N 1/22

(52) U.S. Cl. 702/19; 73/863.01

(57) ABSTRACT

The automatic analyzer is provided with a function of, based on selection of the output result targeted by a user from among all printed results output by the automatic analyzer, proceeding to a start page of the targeted output result so that the targeted output result is displayed, on a screen for displaying a list including a title of printing, the date of printing, a range of pages, and a user's check indicating whether or not the user has checked the result. The automatic analyzer is also provided with a function of enabling a user to add a check indicating whether or not the verification/confirmation of data has been performed, the check date, a checker, a comment, and the like, to the measurement result targeted by the user, and the like, on a screen displaying the result.

### BACKGROUND OF THE INVENTION

1. Field of the Invention

The present invention relates to an automatic analyzer for performing qualitative/quantitative analysis of biological samples such as blood and urine, and more particularly to an automatic analyzer that includes a display unit for displaying information including the measurement result of biological samples.

2. Description of the Related Art

Automatic analyzers, which perform qualitative/quantitative analysis of specific components included in biological samples such as blood and urine, are coming into remarkably wide use mainly in large-scale hospitals, and clinical laboratory test centers, in which it is necessary to handle a large number of samples/items in a short period of time. For this reason, the high measurement precision, high repeatability, high throughput per unit time, and low running costs are required for such automatic analyzers.

In order to satisfy many user needs, for example, automatic analyzers are provided with: a mode in which the measurement result of a biological sample is printed by an actual printer in real time; and a paperless mode in which a printing image is merely displayed on a display unit without actually printing the printing image. The output mode used differs for each facility.

In addition, the automatic analyzers have various kinds of maintenance functions of maintaining the performance of the automatic analyzers. Moreover, the automatic analyzers have various kinds of real-time/batch printing functions of printing the result of executed maintenance, a situation of a reagent used for analysis, the result of calibration, precision control data, and the like, if necessary. The print output results are summarized, and are then displayed in time series as a printing image on a result display screen.

Incidentally, for example, an automatic analyzer as disclosed in JP-A-8-211064 is known as this kind of automatic analyzer.

### SUMMARY OF THE INVENTION

The conventional automatic analyzer displays various kinds of output results, which are acquired by the automatic analyzer, in time series as one printing image. Therefore, in the present situation, it is not possible to know the result included in each page without actual browsing of each page in succession.

Accordingly, the output result targeted by a user may be acquired based on the paging operation of the user, which causes a hindrance to quick operation required in the clinical laboratory test.

In addition, for the data verification/confirm of the measurement result, the user prints the targeted output result from a result output screen to paper for printing, and then confirms/verifies the measurement result of each sample on the printing paper. Accordingly, in the present situation, the user is forced to input a check indicating whether or not the measurement result has been checked, and a comment if necessary, and then the user files and saves the print if necessary.

An object of the present invention is to provide an automatic analyzer that is capable of decreasing a load placed on a user who confirms/verifies the measurement result of biological samples, the maintenance execution result, and the like, and that enables the quick clinical laboratory test operation.

In order to achieve the above-described object, according to one aspect of the present invention, there is provided an automatic analyzer for analyzing a biological sample, the automatic analyzer comprising: a display unit for displaying a list of pieces of information including measurement results of the biological sample; and control means for performing control so that, on each piece of information including the measurement results of the biological sample, information as to when and who has executed verification/confirmation of data can be displayed on the display unit.

In addition, an automatic analyzer may also be provided with a function of enabling a user to add a check indicating whether or not the user has checked the result, the check date, a checker, a comment, and the like, to the measurement result of each biological sample, and the like, on a display unit including a print result screen for displaying the measurement result of the biological sample as a time-series printing image, the measurement result being output by the automatic analyzer.

Moreover, the automatic analyzer may also be provided with various functions based on selection of the output result targeted by a user from among all printed results output by the automatic analyzer, on a display unit that includes a screen for displaying a list including a title of printing, the date and time of printing, an operator ID, a range of page, and user's check indicating whether or not the user has checked the measurement result. Examples of the various functions include a function of proceeding to a start page of the targeted output result so that the targeted output result is displayed; a function of extracting only the output result targeted by the user so that the targeted output result is displayed; a function of printing only the output result targeted by the user on printing paper by a printer; and a function of enabling a user to save only the output result targeted by the user in an outside medium as a printing image by an external storage device.

Furthermore, the automatic analyzer may also be provided with a function of, by selecting and registering only print results required for a user among all printed results output by the automatic analyzer, enabling the user to display only a list of the selected print results so that it is easier for the user to search for the print results targeted by the user.

According to the present invention, a user can easily display a targeted result display screen, and can perform on a screen all confirmation/verification work of the measurement result acquired up to that time. In addition, it is also possible to print the check result displayed on the screen just as it is, or to save the check result in an outside medium just as it is, if necessary. Therefore, it is expected that an improvement in work efficiency can be achieved.

### BRIEF DESCRIPTION OF THE DRAWINGS

FIG. 1 is a diagram schematically illustrating an overall configuration of an automatic analyzer;

FIG. 2 is a diagram illustrating a print result screen, and a menu screen that is displayed from the print result screen;

FIG. 3 is a diagram illustrating a print result screen on which the measurement result of biological samples is displayed;

FIG. 4 is a diagram illustrating a processing method for achieving the present invention; and

FIG. 5 is a diagram illustrating a menu setting screen that is displayed from a print result menu screen.

### DETAILED DESCRIPTION OF THE PREFERRED EMBODIMENTS

Embodiments of the present invention will be described with reference to accompanying drawings as below.

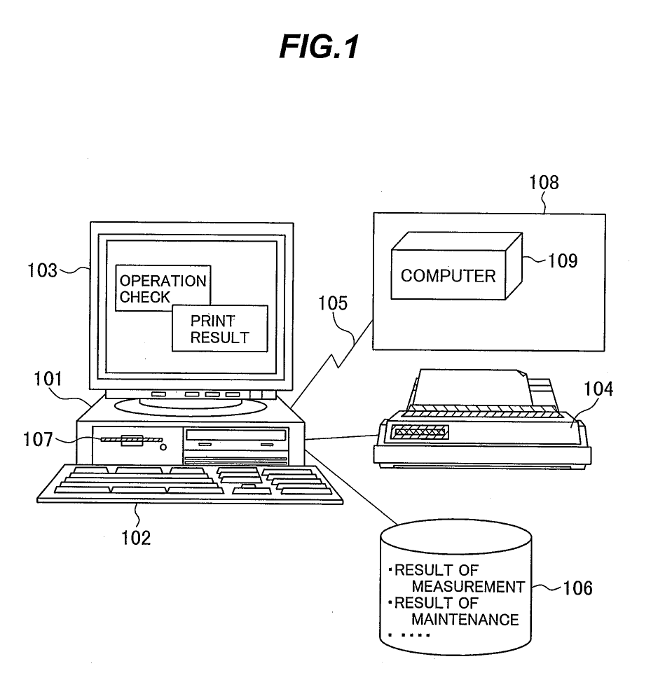

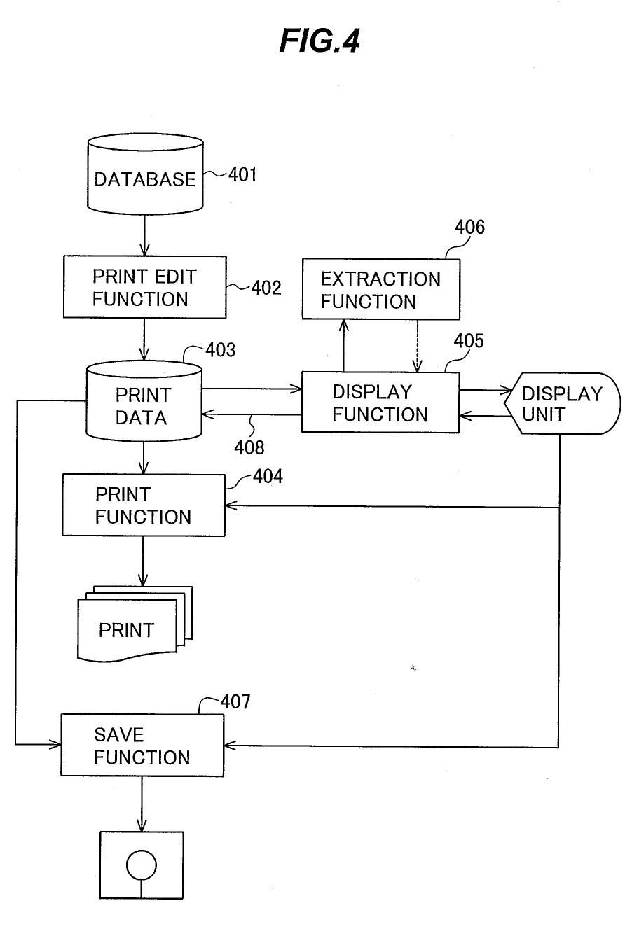

FIG. 1 is a diagram schematically illustrating a configuration of an automatic analyzer. FIG. 4 is a diagram illustrating a processing method for achieving the present invention.

In FIG. 1, reference numeral 101 denotes an operation unit. The operation unit is a computer that is equipped with peripheral devices including: a keyboard 102 for inputting data; a display unit 103 for displaying data; a printer 104 for printing data; an interface 105 through which a connection to an analyzing unit is made; an internal storage device 106 for storing data; and an external storage device 107 for storing data.

Reference numeral 108 denotes one or more analyzing units. The analyzing unit 108 is connected to the operation unit 101 through the interface 105. The analyzing unit 108 executes measurements of biological samples, the maintenance operation, and the like, according to an operation instruction received from the display unit 103. The execution operation is totally controlled by the computer 109.

The result of the executed measurements of the biological samples, the result of the executed maintenance, and the like, are stored in the storage device 106 of the operation unit 101 through the interface 105. In addition, the measurement result and the maintenance execution result are edited in an actual print format to form print data 403 (a print edit function 402 shown in FIG. 4). The print data 403 is then displayed on the display unit 103, or printed by the printer 104 (a print function 404).

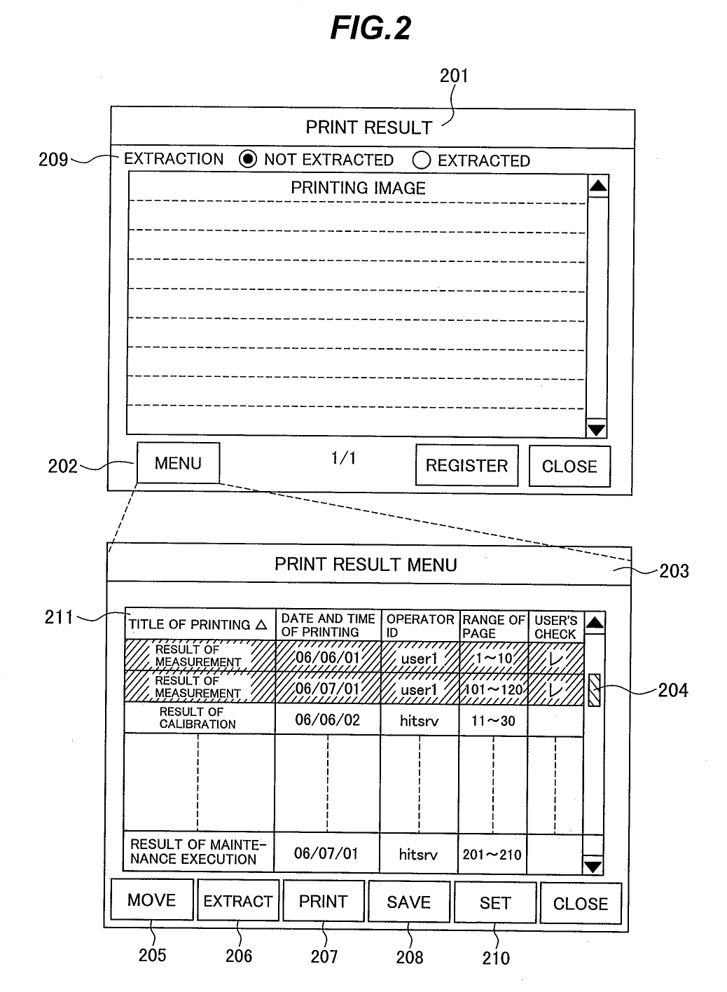

FIG. 2 is a diagram illustrating a print result screen, and a menu screen that is displayed from the print result screen.

The print result screen 201 displays the measurement result of the biological samples, the maintenance execution result, and the like, as a time-series printing image. In addition, a print result menu screen 203, which is displayed by pressing a menu button 202 of the print result screen 201, displays a list (a display function 405) including all of the printed results output by the automatic analyzer. For example, the displayed list includes a title of printing, the date and time of printing, an operator ID, a range of page, and a user's check indicating whether or not a user has checked the result.

In the course of creating print data 403 from a database 401 stored in the storage device 106, each print data about the title of printing, the date and time of printing, and the range of page is successively collated, followed by being stored in a database as list information.

On the print result menu screen 203, a user is allowed to select a plurality of target print results (reference numeral 204). For the selected print results, the user can execute each function by pressing each corresponding button of “Move” 205, “Extract” 206, “Print” 207, or “Save” 208.

In addition, the list displayed on the menu screen can be collated by sorting the fields of the title of printing, the date and time of printing, the operator ID, the range of page, and the user's check (reference numeral 211).

When the “Move” button 205 is pressed, the print result menu screen 203 is closed, and then a start page of the print result selected by the user is displayed on the print result screen 201.

When the “Extract” button 206 is pressed, the print result menu screen 203 is closed, and then the print result screen 201 is displayed with only the print result selected by the user extracted (an extraction function 406). Incidentally, in this case, an extraction radio button 209 of the print result screen 201 is switched to “extracted”. If the screen is returned to a screen with no extraction, the extraction radio button 209 is switched to “not extracted”.

When the “Print” button 207 is pressed, only the print result selected by the user is printed on printing paper by the printer 104 (the print function 404).

When the “Save” button 208 is pressed, only the print result selected by the user is saved in an outside medium as a printing image by the external storage device 107 (a save function 407).

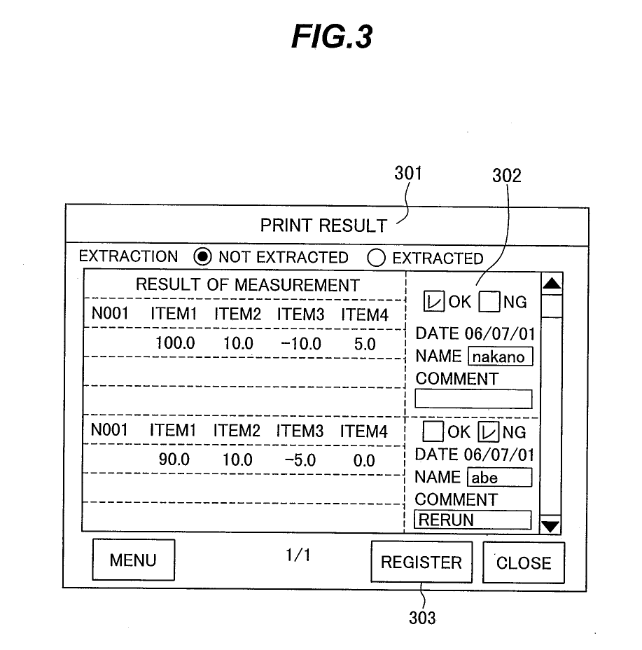

FIG. 3 is a diagram illustrating a print result screen 301 on which the measurement result of biological samples is displayed through the operation from the print result menu screen 203.

An area 302 is displayed on the print result screen 301 together with the actual measurement result. The area 302 is used to input a check mark indicating whether or not data has been checked, the check date, a checker, a comment, and the like.

When a user inputs data into the area 302, the color of a “Register” button 303 changes to prompt the user to register the data. When the “Register” button 303 is pressed, the data, which has been inputted by the user up to this time, is collectively registered (reference numeral 408). Then, the color of the “Register” button is returned. As a result of this operation, the print result in question is displayed as “checked” on the print result menu screen 203.

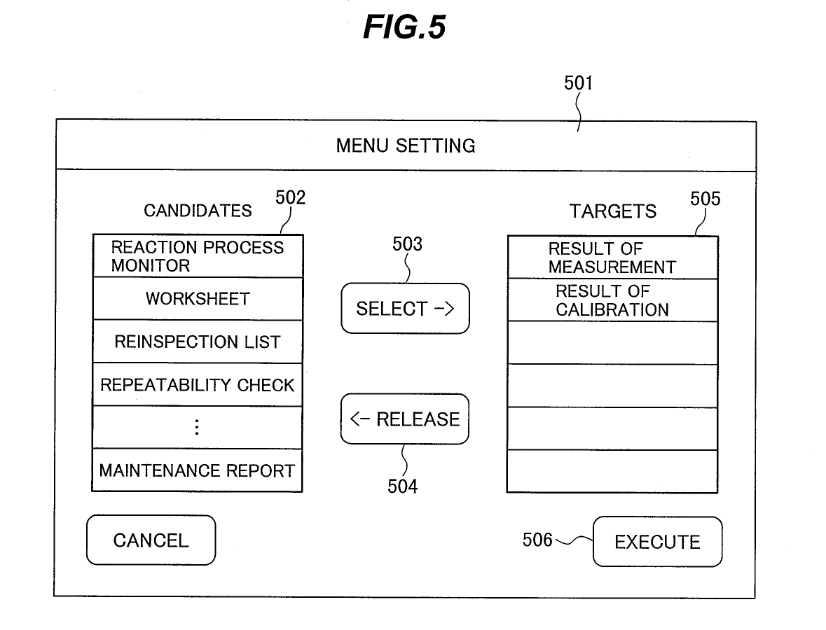

FIG. 5 is a diagram illustrating a menu setting screen that is displayed by pressing a “Set” button 210 of the print result menu screen shown in FIG. 2.

On the menu setting screen 501, print items are displayed in a candidate list 502. An arbitrary print item is moved to a print target list 505 by use of a “Select” button 503. On the other hand, an arbitrary print item included in the print target list 505 is excluded from the print target list 505 by use of a “Release” button 504. In actuality, the operation of selection/cancel causes the color of a “Return” button 506 to change and then pressing the “Return” button 506 closes the menu setting screen 501. Then, the print result menu screen 203 displays only the print items selected on the menu setting screen 501.

What is claimed is:

1. An automatic analyzer for analyzing a biological sample, said automatic analyzer comprising:
a display unit for displaying a list of pieces of information including measurement results of the biological sample; and
control means for performing control so that, on each piece of information including the measurement results of the biological sample, information as to when and who has executed verification/confirmation of data can be displayed on the display unit.

2. The automatic analyzer according to claim 1, further comprising:
input means for inputting information as to when and who has executed verification/confirmation of data.

3. The automatic analyzer according to claim 1, further comprising:
selection means for, on the display unit, selecting output results targeted by a user from among the listed pieces of information, and
control means for performing control so that the information selected by the selection means is displayed.

4. The automatic analyzer according to claim 3, further comprising:
control means for performing control so that only information selected by the selection means is displayed on the display unit.

5. The automatic analyzer according to claim 3, further comprising:
control means for performing control so that only information selected by the selection means is printed by a printer.

6. The automatic analyzer according to claim 3, wherein:
only information selected by the selection means can be saved in an outside medium as a printing image through an external storage device.
